(19) United States
(12) Patent Application Publication
(10) Pub. No.: US 2024/0325341 A1
(43) Pub. Date: Oct. 3, 2024

(54) ANTI-BACTERIAL EFFECT OF HALOGENATED FLUORESCEINS AGAINST COLISTIN-RESISTANT GRAM-NEGATIVE BACTERIA

(71) Applicants: Provectus Pharmatech, Inc., Knoxville, TN (US); University of Tennessee Research Foundation, Memphis, TN (US)

(72) Inventors: Michio KUROSU, Knoxville, TN (US); Dominic Rodrigues, Knoxville, TN (US); Edward V. Pershing, Knoxville, TN (US); Bruce Horowitz, Knoxville, TN (US); John Lacey, Knoxville, TN (US); Eric A. Wachter, Oak Ridge, TN (US)

(73) Assignees: Provectus Pharmatech, Inc., Knoxville, TN (US); University of Tennessee Research Foundation, Memphis, TN (US)

(21) Appl. No.: 18/615,444

(22) Filed: Mar. 25, 2024

Related U.S. Application Data
(60) Provisional application No. 63/455,501, filed on Mar. 29, 2023.

(51) Int. Cl. A61K 31/352; A01N 43/16; A01P 1/00; A61N 5/06; A61P 31/04

(52) U.S. Cl. CPC A61K 31/352; A01N 43/16; A01P 1/00; A61N 5/062; A61P 31/04; A61N 2005/0651; A61N 2005/0663

(57) ABSTRACT

of treating Gram-negative bacteria that also exhibit resistance to the anti-bacterial compound colistin (MIC≥50 mg/mL) except for Burkholderia, Proteus, and Serratia that comprises contacting the bacteria with an aqueous pharmaceutical composition containing a rose bengal (RB) compound of Formula I, below, dissolved or dispersed therein at a concentration of about 0.01 to about 15 mg/mL and irradiating those contacted bacteria with light of the wavelength about 500 nm to about 600 nm for a time period of about 1 to about 10 minutes to provide a light dose of about 16 to about 160 J/cm², treat and kill the irradiated bacteria

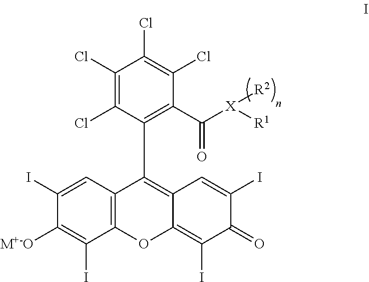

wherein X, R¹, R² and M⁺ are defined within.

### CROSS-REFERENCE TO RELATED APPLICATIONS

This application claims the benefit of U.S. Provisional Patent Application No. 63/455,501, filed Mar. 29, 2023, entitled ANTI-BACTERIAL EFFECT OF HALOGENATED FLUORESCEINS AGAINST COLISTIN-RESISTANT GRAM-NEGATIVE BACTERIA, the entire contents of which are incorporated herein by reference.

### DESCRIPTION

### Background Art

Multidrug resistant (MDR) Gram-negative bacteria have a significant impact on morbidity and mortality [1-4]. Colistin (polymyxin E), an antibiotic first discovered almost 60 years ago, is effective against most multidrug-resistant Gram-negative bacteria [5,6]. Except for patients with cystic fibrosis [7-10], it has not been used widely since the early-1980s because of its nephrotoxicity. However, it has been reintroduced in clinical practice as a last-line drug to treat severe nosocomial infections caused by multidrug resistant Gram-negative bacteria [11].

Many studies have shown that the prevalence of colistin-resistance has increased rapidly among Enterobacteriaceae [12-14]. Some clinical isolates of these bacteria, including Pseudomonas aeruginosa, Acinetobacter baumannii, Escherichia coli, Salmonella spp., and Klebsiella spp. have acquired resistance against colistin [15-20]. Thus, colistin resistance is considered a serious public health problem due to a lack of alternative therapeutic options.

Interestingly, other Pseudomonadota (Proteobacteria), such as Serratia spp., Proteus spp., and Burkholderia spp. exhibit natural resistance to colistin [21]. Colistin-resistant Gram-negative bacteria are also often cross-resistant to other anti-bacterial agents [22, 23]. For example, meropenem, an ultra-broad spectrum carbapenem, is effective against drug-susceptible K. pneumoniae, P. aeruginosa, and A. baumannii at low concentrations (0.03-6.25 g/mL), whereas colistin-resistant strains of P. aeruginosa, A. baumannii, and K. pneumoniae reduce their susceptibility against meropenem and other antibiotics [24, 25].

Colistin-resistant K. pneumoniae strains are difficult to treat with currently available drug arsenals [26, 27]. Although many studies have been devoted to optimizing the use of currently available agents or identifying any combination thereof, none of the pipeline drugs have been directed to solve drug-resistant bacteria associated with colistin resistance [28-33]. Thus, drugs effective against colistin-resistant strains or methods to prevent the acquisition of colistin resistance during treatment are needed.

U.S. Pat. Nos. 8,530,675, 9,273,022, and 9,422,260 to Singer et al. describe and claim the synthesis of highly purified rose bengal, as well as similarly purified compounds containing different halogen substituents and different numbers of those halogen substituents, as well as their lactone forms. The highly purified rose bengal used in the above patents was a clinical-stage formulated rose bengal (HB-RBf) composition containing 10% rose bengal w/v in 0.9 percent aqueous sodium chloride (NaCl) provided for study by Provectus Biopharmaceuticals, Inc., of Knoxville, TN) under the name PV-10®. Rose bengal compounds are collectively referred to herein as “halogenated xanthenes,” and more specifically as “halogenated fluoresceins.”

RB dye has been clinically investigated for the treatment of melanoma and other solid cancers [36]; and PCT/US22/05407, particularly when injected directly into a cancerous lesion. Photodynamic anti-bacterial properties of RB have been sporadically reported and PCT/US22/054076.

Dees et al. U.S. Pat. No. 8,974,363 teache the use of a topical formulation of RB at 10-100 μM (i.e., 10 μg/mL to 100 μg/mL) in conjunction with green light irradiation in the 500-600 nm wavelength band against Gram-positive and Gram-negative antibiotic-resistant bacteria without specifics as to the bacterial strains used, the light source, its intensity or duration of irradiation.

We have screened in-house library molecules, including FDA-approved and non-approved anti-bacterial agents, against two Burkholderia spp., one Proteus sp., one Serratia sp., colistin-resistant Pseudomonas aeruginosa, and a wild-type Pseudomonas aeruginosa (a reference bacterial strain). We identified that rose bengal (RB; 4,5,6,7-tetrachloro-2′,4′,5′,7′-tetraiodo-fluorescein) displays strong bactericidal activity (0.05-6.25 □g/mL) against colistin-resistant Gram-negative strains under a fluorescent light (see, Table 1 hereinafter and). However, RB did not show appreciable anti-bacterial activity (MIC<12.5 □g/mL) against wild-types of the other Gram-negative bacteria examined (E. coli, A. baumannii, K. pneumoniae, and P. aeruginosa).

RB's anti-bacterial characteristics discussed above led us to investigate efficacy of RB against Gram-negative bacteria that acquired colistin resistance. To validate the bactericidal effect of RB against colistin-resistant strains, we prepared medium- to high-colistin-resistant strains of E. coli, A. baumannii, K. pneumoniae, P. aeruginosa, Burkholderia spp., S. enterica Typhimurium, and Serratia sp. Herein, we report susceptibility of the pharmaceutical grade RB [high-purity RB formulation (HP-RBf)] against these colistin-resistant Gram-negative bacteria.

### BRIEF SUMMARY OF THE INVENTION

The present invention contemplates the combined use of a rose bengal derivative in conjunction with irradiation of Gram-negative bacteria that also exhibit resistance to the anti-bacterial compound colistin (MIC≥50 mg/mL) with a light of the wavelength about 500 nm to about 600 nm for a time period of about 1 to about 10 minutes to treat and kill the irradiated bacteria.

In one embodiment, the present invention contemplates the combined use of a rose bengal derivative in conjunction with irradiation of Gram-negative bacteria that also exhibit resistance to the anti-bacterial compound colistin (MIC≥50 mg/mL except for Burkholderia, Proteus, and Serratia with light of the wavelength about 500 nm to about 600 nm for a time period of about 1 to about 10 minutes to treat and kill the irradiated bacteria.

In one aspect, Gram-negative colistin-resistant bacteria, and in an embodiment ones other than Burkholderia, Proteus, and Serratia, are treated in a method that includes the steps of contacting the bacteria with an aqueous pharmaceutical composition containing a rose bengal (RB) compound of Formula I, below, dissolved or dispersed therein at a concentration of about 0.01 to about 15 mg/mL; and irradiating those contacted bacteria with light of the wavelength about 500 nm to about 600 nm for a time period of about 1 to about 10 minutes to provide a light dose of about 16 to about 160 J/cm². The RB compound of Formula I is shown below:

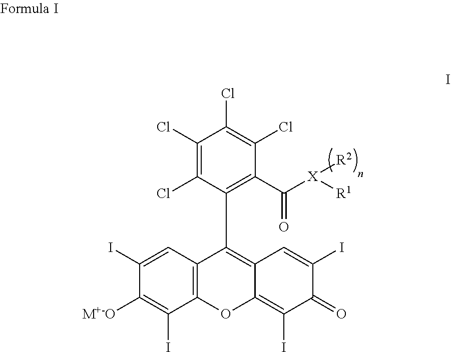

wherein X is oxygen or nitrogen, “n” is zero or one such that when X is oxygen, n is zero and R² is absent, whereas when X is nitrogen, n is one and R² is present. When X is oxygen, R¹ is selected from the group consisting of hydrogen (H), M⁺ that is a pharmaceutically acceptable cation, C₁-C₄ alkyl, and an aromatic ring-containing substituent as defined hereinafter. When X is nitrogen, R¹ and R² are the same or different and are selected from the group consisting of hydrogen, C₁-C₄ alkyl, or together with amido nitrogen atom form a 5- or 6-membered ring and an aromatic ring substituent. The aromatic ring substituent is a single ring containing 5- or 6-members, or a 5,6- or 6,6-fused aromatic ring system, and the aromatic ring or ring system substituent can contain 0, 1, or 2 hetero ring atoms that are independently nitrogen, oxygen, or sulfur.

Structural formulas of exemplary aromatic ring substituents are set out below:

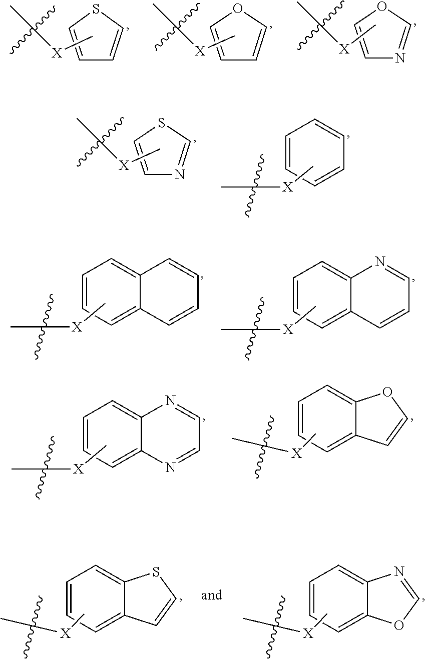

where

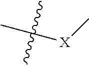

is

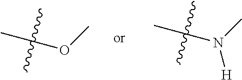

providing an ester or a monosubstituted amine, respectively.

In one preferred embodiment, the method is carried out in an area where surgery is performed to inhibit post-surgical infection by Gram-negative, colistin-resistant bacteria. A contemplated aqueous pharmaceutical composition containing a RB compound of Formula I can be applied to surfaces of the surgical area by spraying or swabbing or any other convenient method, as can the intracorporeal and extracorporeal areas of the surgical patient's body, as well as the surgical instruments, a surgeon's hands, the hands of others attending the procedure and the various instruments present in the surgical area. One or more lights emitting the before-noted amount of light in the wavelength about 500 nm to about 600 nm can be used to illuminate the entire surgical area or a lesser area as desired.

### BRIEF DESCRIPTION OF THE DRAWINGS

In the drawings forming a portion of this disclosure

FIG. 1, in six panels as FIGS. 1A, 1B, 1C, 1D, 1E, and 1F, are graphs that illustrate time-kill kinetics of HP-RBf (10% RB in saline) against colistin-resistant [colistin^{R}] Gram-negative bacteria under the fluorescent light (17 W, 63.8 cm², 0-40 J/cm²). A 5-times the MIC concentration was applied: E. coli ATCC35218 colistin^{R}: MIC 400 mg/mL (colistin); P. aeruginosa ATCC27853 colistin^{R}: MIC>400 mg/mL (colistin); K. pneumoniae ATCC 19606 colistin^{R}: MIC 100 mg/mL (colistin); and A. baumannii ATCC BAA-1800 colistin^{R}: MIC 400 mg/mL (colistin).

### DETAILED DESCRIPTION OF PREFERRED EMBODIMENTS

The present invention contemplates a method of treating Gram-negative bacteria that also exhibit resistance to the anti-bacterial compound colistin (MIC≥50 mg/mL), and in one embodiment, Gram-negative bacteria that also exhibit resistance to the anti-bacterial compound colistin except for Burkholderia, Proteus, and Serratia, that comprises contacting the bacteria with an aqueous pharmaceutical composition containing a rose bengal (RB) compound of Formula I, below, dissolved or dispersed therein at a concentration of about 0.01 to about 15 mg/mL and irradiating those contacted bacteria with light of the wavelength about 500 nm to about 600 nm for a time period of about 1 to about 10 minutes to provide a light dose of about 16 to about 160 J/cm², to treat and kill the irradiated bacteria.

A contemplated RB compound has the structural formula (Formula I) below, where X is O (oxygen) or N (nitrogen), and “n” is zero or one.

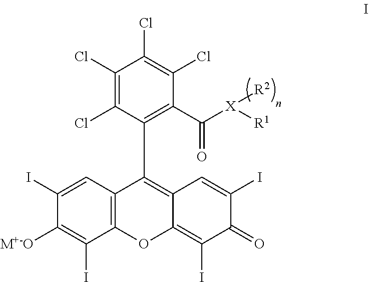

When X is oxygen, n is zero and absent so that the RB compound is: a) rose bengal where —X—R¹ is —O—H, b) a pharmaceutically acceptable salt of RB where —X—R¹ is —O⁻M⁺ and where M⁺ is a pharmaceutically acceptable cation, c) a C₁-C₄ alkyl ester, or d) an aromatic ester as defined below.

Alternatively, when X is a nitrogen atom, n is 1, and R² is present along with R¹. As such, R¹ and R² can be the same or different, and C(O)NR¹R² is an amide whose nitrogen atom is a) unsubstituted [—X—(R¹R²) and both R¹ and R² are hydrogen (H)], is b) substituted with one or two C₁-C₄ alkyl groups or together with the amido nitrogen atom form a 5- or 6-membered ring, or is c) an aromatic amide that is preferably monosubstituted in that R¹ is hydrogen and R² is the aromatic substituent discussed below.

For ease of description, an aromatic ester or aromatic amide are collectively referred to as an aromatic derivative. As such, those derivatives are formed from an alcohol or amine, preferably monosubstituted, having a single 5- or 6-membered aromatic ring or a 5,6- or 6,6-fused aromatic ring system that contains 0, 1, or 2 hetero ring atoms that are independently nitrogen, oxygen or sulfur.

Illustrative examples of such aromatic alcohol ester portions are shown and named below, where O is an oxygen atom and line-O indicates the ring-oxygen can be from any available carbon of the ring, and the O-line crossed by a wavy line indicates that the depicted alkoxy group is a portion of another molecule, the esterified RB molecule.

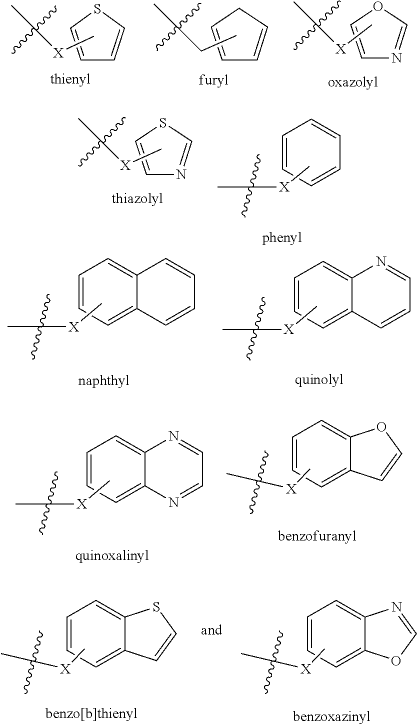

where

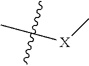

is

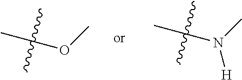

providing an ester or a monosubstituted amine, respectively.

Rose bengal (RB) is a preferred RB compound, and its disodium salt, rose bengal disodium (RBD), is the most preferred RB compound. These compounds are used illustratively herein for the group of RB compounds.

The chemical name for rose bengal is 4,5,6,7-tetrachloro-2′,4′,5′,7′-tetraiodo-fluorescein. A preferred form, rose bengal disodium (RBD), has the following structural formula:

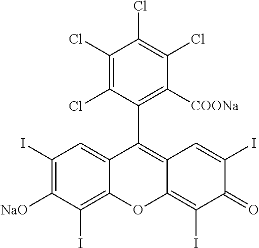

Certain details of this preferred embodiment for a contemplated composition are described in U.S. Pat. Nos. 5,998,597, 6,331,286, 6,493,570, and 8,974,363, whose disclosures are incorporated by reference herein in their entireties. The above patents describe the use of RBD to kill cancer cells.

In one aspect, mammalian cells, such as those of humans, which are infected by the Gram-negative colistin-resistant bacteria, and the bacteria are contacted by RB compound that is taken-up by the infected cells. In another aspect, the Gram-negative colistin-resistant bacteria are present on a surface that is to be disinfected.

The light can be administered, along with a contemplated RB-containing pharmaceutical composition in which a contemplated RB compound is present, contacting the infected area at a concentration of about 0.01 to about 15 mg/mL, and preferably about 0.2 to about 3.1 mg/mL, dissolved or dispersed in a pharmaceutically acceptable diluent. The light can also be administered shortly after application of the pharmaceutical composition to contact the bacteria or mammalian cells containing the bacteria, preferably within about 2 to about 5 minutes. The bacteria can be treated within infected mammalian cells or when present on a surface such as an examination or surgical table.

Although seemingly similar to prior treatments, it is believed that the concentrations of the RB compound are less than those used previously and that the light amount is about one-tenth or less than that of a prior method. In addition, this treatment is directed at Gram-negative bacteria that are also resistant to colistin treatment, other than Burkholderia, Proteus, and Serratia.

Colistin-resistance, in its various grammatical forms, is used herein to mean that the MIC is about equal to or greater than (≥) a colistin concentration of about 50 mg/mL. In most instances, the MIC is ≥ about 100 mg/mL and is often ≥ about 400 mg/mL. As a consequence, less of the anti-bacterial RB compound can be used, and lower-priced lighting can be used, thereby making the process more practicable.

The infected area treated with RB is irradiated for a time period of about 1 to about 10 minutes, and more preferably about 2 to about 5 minutes. Such irradiation provides a light dose of about 16 to about 160 J/cm², and more preferably, provides a light dose of about 32 to about 80 J/cm².

### Pharmaceutical Halogenated Xanthene Composition

A contemplated liquid composition can be formulated for oral administration, parenteral administration or topical administration. Illustrative halogenated xanthene (fluorescein) compositions are illustrated by reference to the use of rose bengal disodium, the preferred halogenated xanthene compound.

Turning first to a parenteral or topical treatment composition, the previously noted PV-10® composition is illustrative of a parenterally-administrable pharmaceutical composition containing the particularly preferred RB compound, rose bengal disodium. Delivery of the halogenated fluorescein component of a contemplated composition is most favorable when the composition has a pH value close to physiologic pH (i.e., approximately pH 7), and especially when the pH value is greater than about 4, thereby assuring that a halogenated fluorescein remains in dibasic form in the composition.

Thus, in a preferred embodiment, the pH value of the composition is about 5 to about 9, and more preferably about 6 to about 7.5, and most preferably about pH 6.5 to about pH 7.4. At these pH values, the halogenated fluoresceins typically remain in dibasic form rather than the lactone that forms at low pH values.

An RB compound such as rose bengal is dibasic, having pKa values of 2.52 and 1.81. pKa value determinations for several contemplated halogenated fluoresceins can be found in Batsitela et al., Spectrochim Acta Part A 79 (5): 889-897 (September 2011).

A hydrophilic vehicle is one preferred medium for the medicament to maximize preference for partitioning of the halogenated fluorescein component into tissue, particularly for RB compounds in the acid and/or salt form. Accordingly, in a preferred embodiment, the vehicle contains a minimum of non-hydrophilic components that might interfere with such partitioning. Thus, a preferred formulation of the composition contains RB, or RB disodium, that is particularly preferred in a hydrophilic, preferably water-containing vehicle.

When administered parenterally, other than in a suppository, RB compound-containing pharmaceutical composition preferably includes a water-soluble electrolyte comprising at least one cation selected from the group consisting of sodium, potassium, calcium, and magnesium and at least one anion selected from the group consisting of chloride, phosphate, and nitrate. The electrolyte is preferably at a concentration of about 0.1% (w/v) and about 2% (w/v).

Alternately, the electrolyte is present at a level sufficient to provide an osmolality of greater than approximately 100 mOsm/kg (Milliosmoles Per Kilogram Of Water) up to about 600 mOsm/kg. More preferably, the osmolality of the medicament composition is greater than 250 mOsm/kg, and most preferably approximately 300-500 mOsm/kg.

The electrolyte is preferably sodium chloride. The electrolyte is preferably present at a concentration of about 0.5 to about 1.5%, and even more preferably at a concentration of about 0.8 to about 1.2%, and most preferably at a concentration of approximately 0.9%, as is present in physiological saline.

The aqueous medium (diluent) of the composition is preferably only water that meets the criteria for use in injection. Up to about 20 percent by volume of the diluent can be one or more C1-C6 mono- or polyhydric alcohols such as methanol, ethanol, propanol, isopropanol, butanol, sec-butanol, glycerol, ethylene glycol, propylene glycol, 1, 2-butanediol, 2, 3-butanediol, erythritol, threitol, trimethylolpropane, sorbitol and the like. More preferably, an alcohol is present in a contemplated composition at less than about 10 percent by volume of the diluent, and more preferably at less than about 5 percent by volume.

The terms “physiologically acceptable salt” and “pharmaceutically acceptable salt” in their various grammatical forms refer to any non-toxic cation such as an alkali metal, alkaline earth metal, and ammonium salt commonly used in the pharmaceutical industry, including the sodium, potassium, lithium, calcium, magnesium, barium, and protamine zinc salts, which can be prepared by methods known in the art. A contemplated cation provides a water-soluble RB salt. Preferably, the salts are sodium, potassium, and calcium in either the mono or dibasic salt form. The reader is directed to Berge, J. Pharm. Sci. 1977 68 (1): 1-19 for lists of commonly used physiologically (or pharmaceutically) acceptable acids and bases that form physiologically/pharmaceutically acceptable salts with pharmaceutical compounds.

The pH value of the RB-containing pharmaceutical composition can be regulated or adjusted by any suitable means known to those of skill in the art. The composition can be buffered, or the pH value can be adjusted by the addition of an acid, a base, or the like. As RB, or physiologically acceptable salts thereof, are weak acids, depending upon their concentration and/or electrolyte concentration, the composition's pH value may not require a buffer and/or pH value-modifying agent. It is especially preferred, however, that the composition be free of buffer, allowing it to conform to the biological environment once administered.

It is also preferred that the pharmaceutical composition not include any preservatives, many of which can deleteriously interfere with the pharmaceutical composition or formulation thereof or may complex or otherwise interact with or interfere with the delivery of the RB compound-containing composition active component. To the extent that a preservative is used, imidurea is a preferred preservative as it does not interact with RB compounds, either in the pharmaceutical composition or upon administration.

A contemplated liquid pharmaceutical composition can also be adapted for oral administration to the mammalian subject to be treated. In a preferred aspect, the RB compound, as previously discussed, is dissolved or dispersed in an aqueous diluent when administered to a mammalian subject. It is more preferred that the aqueous diluent be free of tonicity agents except for those sugars and/or buffering agents present as flavorants.

Up to about 20 percent by volume of the diluent can be one or more C₁-C₆ mono- or polyhydric alcohols as was previously discussed. More preferably, an alcohol is present in a contemplated composition at less than about 10 percent by volume of the diluent, and more preferably at less than about 5 percent by volume.

A topically applied liquid composition is also contemplated. One such liquid composition that is undergoing clinical trials for the treatment of psoriasis is a developmental medicament called PH-10® by Provectus Biopharamceuticals, Inc. of Knoxville, TN. This medicament contains RBD present at a concentration of 0.001 to 0.01% w/v dissolved or dispersed in an aqueous diluent, along with at least one builder present at a level sufficient to provide a viscosity of 10-1000 cps to the medicament, sodium chloride as an electrolyte present at a concentration of 0.9% w/v of at a level sufficient to provide an osmolality of 100 mOsm/kg to 500 mOsm/kg to the medicament. This medicament is described and claimed in, e.g., U.S. Pat. No. 8,974,363.

### Treatment Method

A contemplated treatment method comprises contacting Gram-negative bacteria that exhibit resistance to the anti-bacterial compound colistin (MIC≥50 mg/mL). In one embodiment, except for Burkholderia, Proteus, and Serratia, with a composition containing an anti-Gram-negative bacterial amount of an RB compound while those bacterial cells are irradiated with light having a wavelength of about 500 nm to about 600 nm for a time period of about 1 to about 10 minutes to treat and kill the irradiated bacteria. A preferred wavelength for that irradiation is about 500 to about 575 nm and, more preferably about 510 to about 550 nm. A contemplated RB compound-containing composition is considered to provide an effective treatment if the MIC determined, as discussed herein, is about 10 mg/mL or less.

In one embodiment, the Gram-negative bacterial cells are present on or in (e.g., infecting) a subject mammal. Illustratively, a subject mammal can have a dermatological Gram-negative bacterial infection such as that of Klebsiella, Escherichia, or Pseudomonas, or particularly in the case of a topical treatment of an open or surgical wound, as a treatment for a present infection and as a preventative from subsequent Gram-negative bacterial in which the infecting bacteria are also colistin-resistant.

In another embodiment, a surface such as an examination table, the floor and/or walls and/or equipment of a surgical arena can be washed with a contemplated composition as above and irradiated to disinfect or prevent growth of one or more Gram-negative bacteria as discussed above. Washing and irradiation can be carried out concurrently with surgery or other treatments for mammalian subjects to assist in disinfection or improve the overall cleanliness of the mammalian subject and the treatment area.

A treated subject mammal can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Each contemplated composition administration is typically repeated until the treated bacterial disease (infection) is diminished to a desired extent, such as not being detectable. Thus, the administration to a mammalian subject in need can occur a plurality of times within one day, daily, weekly, monthly or over a period of several months to several years as directed by the treating physician.

A Gram-negative bactericidal effective amount of RB is administered to the mammalian subject in need and can be formulated using usual liquid, gel, cream, or other formats. In most instances, the RB is administered with irradiation, preferably using a light source that includes an emission wavelength of about 500 to about 600 nm, and more preferably at about 510 to about 550 nm. Illustrative sources for the light source, its intensity, and its duration are discussed hereinafter.

### Results

### A Pharmaceutical Grade of Rose Bengal Formulation (HP-RBf) Effectively Inhibits the Growth of the Colistin-Resistant Gram-Negative Bacteria

The anti-bacterial activity of rose bengal (RB) has been reported with a commercial grade of rose bengal (80-95% dye content), which contains the known impurities (e.g., transhalogenated substances) produced by the historical manufacturing process [36]. Thus, commercial-grade RB lacks pharmaceutical relevance.

RB applied in this study has a purity of >99.5%, and was synthesized and purified under current good manufacturing practices. High-purity RB was originally formulated in saline (10% RB in saline; HP-RBf) and was further diluted with saline to form the desired concentrations.

Minimum inhibitory concentrations (MICs, μg/mL) were obtained via broth dilution methods under fluorescent (about 23.0 KJ/cm²) and LED light (about 29.0 KJ/cm²) irradiation conditions over a 24-hour time period. Illustratively, that works out to about 0.96 (about 1) KJ/cm² over 60 minutes (min) or about 16.0 J/cm² in one min. For the studies that were made using irradiation times of zero to 30 minutes, using the 17 W, 63.8 cm² fluorescent lamp, the amount of light would correspondingly be about 0-480 J/cm². The about 1 to about 10 minute irradiations discussed in would come out to about 16 to about 160 J/cm².

HP-RBf effectively inhibited the growth of the colistin-resistant Gram-negative bacteria with MIC levels of 0.05-12.5 μg/mL. The bactericidal activity of HP-RBf against the colistin-resistant strains observed in Table 1 below was not noticeably different depending on the light sources; the MIC values were equal or very close for fluorescent and LED lights [35].

TABLE 1
Discovery of a bactericidal molecule against
colistin-resistant bacterial strains.^{a}

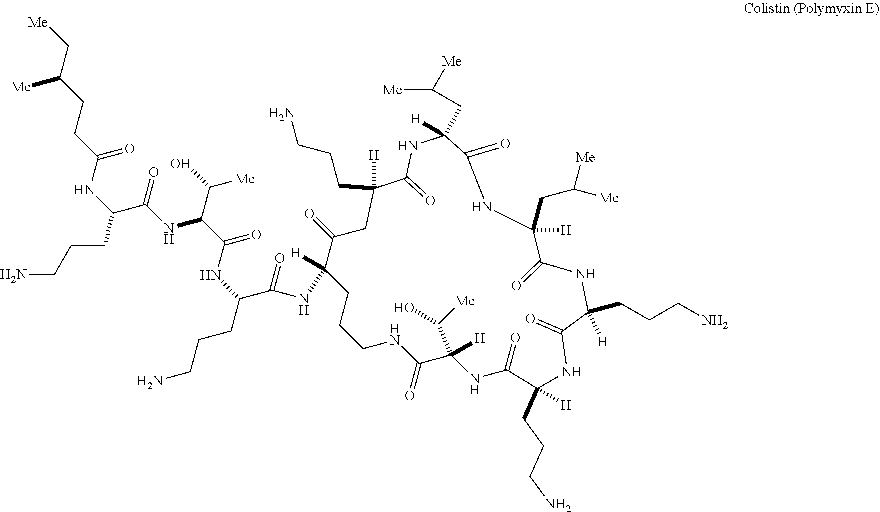

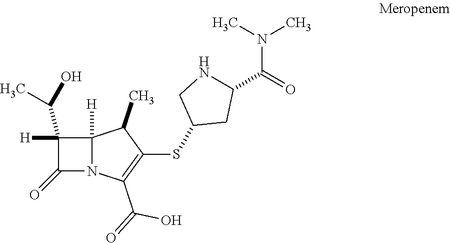

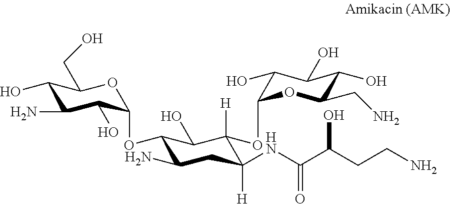

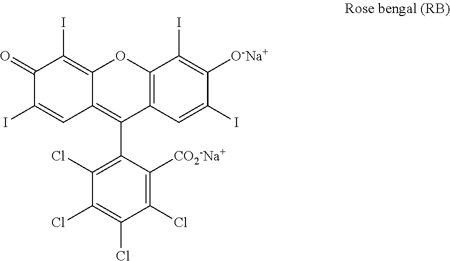

| | Colistin | | | |
|---|---|---|---|---|
| | (Polymyxin | | | |
| | E) | Meropenem | Amikacin | HP-RBf |
| | MIC | MIC | MIC | MIC |
| Bacterial strain^{b} | (_g/mL) | (_g/mL) | (_g/mL) | (_g/mL) ^{d} |
| Pseudomonas | 6.3 | 125 | 100 | 50.0 |
| aeruginosa (ATCC | | | | |
| 27853) | | | | |
| Pseudomonas | >200 | >200 | >200 | 0.05 |
| aeruginosa (ATCC | | | | |
| 27853) colistin- | | | | |
| resistant strain^{c} | | | | |
| Serratia sp. strain | >200 | 0.004 | >200 | 0.8 |
| Ag2 | | | | |

^{a}All studies were in triplicate. The MIC values were determined via OD and colorimetric assays using risazurin or malachite green.
^{b}Bacteria were purchased from ATCC or acquired from BEI Resources.
^{c}Colistin-resistant strains were prepared in the Kurosu laboratory acording to the procedure described hereinafter.
^{d} Light sources used were a 17 W, 63.8 cm² fluorescent light or a 9.5 W, 28.3 cm² LED light. The MIC was determined after 24 h of treatment (23.0 KJ/cm² for fluorecent light or 29.0 KJ/cm² for LED light) .

HP-RBf exhibited bactericidal activity against colistin-resistant E. coli (ATCC 35218, >400 □g/mL against colistin) with the MIC value of 0.05 □g/mL (entry 2, Table 2, below). The susceptibility of meropenem against E. coli was much lower when it acquired colistin resistance (entry 1 vs. 2). E. coli serotype 0157 (wild type) is a major food-borne pathogen that displayed susceptibility to colistin, meropenem, and amikacin (entry 3). We could isolate its colistin-resistant mutant with the MIC level of 100 μg/mL (entry 4).

TABLE 2
HP-RBf susceptibility against colistin-resistant Gram-negative bacterial strains^{a}

| | | | Colistin | | |
|---|---|---|---|---|---|
| | | HP-RBf | (Polymyxin | Meropenem | Amikacin |
| | | MIC | E) MIC | MIC | MIC |
| Entry | Bacterial strain^{b} | (μg/mL)^{d} | (μg/mL) | (μg/mL) | (μg/mL) |
| 1 | Escherichia coli (ATCC 35218) | >100 | 1.6 | <0.8 | 3.1 |
| 2 | Escherichia coli (ATCC 35218) | 0.05 | 400.0 | 25.0 | >100 |
| | colistin-resistant strain | | | | |
| 3 | Escherichia coli serotype O157 (TW07793) | 100 | 1.6 | 1.6 | 1.6 |
| 4 | Escherichia coli serotype O157 (TW07793) | 3.13 | 100.0 | 12.5 | 25.0 |
| | colistin-resistant strain | | | | |
| 5 | Acinetobacter baumannii (ATCC19606) | 50.0 | 3.13 | 100.0 | 16.0 |
| 6 | Acinetobacter baumannii (ATCC19606) | 0.05 | 400.0 | >100 | >100 |
| | colistin-resistant strain | | | | |
| 7 | Acinetobacter baumannii (ATCC BAA1800) | >100 | 0.8 | >100 | 64.0 |
| 8 | Acinetobacter baumannii (ATCC BAA1800) | 0.05 | 400.0 | >100 | >100 |
| | colistin-resistant strain | | | | |
| 9 | Klebsiella pneumoniae (ATCC 19606) | >100 | 6.3 | >100 | 4.0 |
| 10 | Klebsiella pneumoniae (ATCC 19606) | 6.3 | 100.0 | >100 | 64.0 |
| | colistin-resistant strain | | | | |
| 11 | Klebsiella pneumoniae (CRE) CHS 67 | >100.0 | 12.5 | >100 | 16.0 |
| | (NR48569) | | | | |
| 12 | Klebsiella pneumoniae (CRE) CHS 67 | 12.5 | 100.0 | >100 | >100 |
| | (NR48569) colistin-resistant strain | | | | |
| 13 | Pseudomonas aeruginosa (ATCC 27853) | 100.0 | 6.25 | >100 | 32.0 |
| 14 | Pseudomonas aeruginosa (ATCC 27853) - | 6.3 | 50 | >100 | >100 |
| | intermediate colistin-resistant strain | | | | |
| 15 | Pseudomonas aeruginosa (ATCC 27853) - | 0.05 | >400 | >100 | >200 |
| | high colistin-resistant strain | | | | |
| 16 | Pseudomonas aeruginosa MRSN 1356 | 25.0 | 1.56 | >100 | 32.0 |
| | (NR51521) | | | | |
| 17 | Pseudomonas aeruginosa MRSN 1356 | 6.3 | 50.0 | >100 | >100 |
| | (NR51521) colistin-resistant strain | | | | |
| 18 | Pseudomonas aeruginosa MRSN 1380 | 25.0 | 1.56 | 3.13 | 64.0 |
| | (NR51522) | | | | |
| 19 | Pseudomonas aeruginosa MRSN 1380 | 1.6 | 50.0 | 100.0 | >100 |
| | (NR51522) colistin-resistant strain | | | | |
| 20 | Burkholderia multivorans CGD1 (wild type) | 6.3 | 200 | 25.0 | 200.0 |
| 21 | Burkholderia multivorans CGD1 | 0.05 | >400 | >100 | >200.0 |
| | colistin-resistant strain | | | | |
| 22 | Burkholderia thailandensis E264 (Wild type) | 6.3 | 200 | 12.5 | 64.0 |
| 23 | Burkholderia thailandensis E264 | 0.8 | >400 | >100 | >200 |
| | colistin-resistant strain | | | | |
| 24 | Burkholderia cepacia genomovar III, | 6.3 | 200 | 25.0 | >100 |
| | LMG16656 (wild type) | | | | |
| 25 | Burkholderia cepacia genomovar III, | 0.39 | >400 | 100 | >200 |
| | LMG16656 colistin-resistant strain | | | | |
| 26 | Salmonella enterica subsp. enterica | 12.5 | 0.8 | 3.1 | 1.6 |
| | Typhimurium (BAA 2721) (Wild type) | | | | |
| 27 | Salmonella enterica subsp. enterica | 3.1 | 12.5 | 6.25 | >100 |
| | Typhimurium (BAA 2721) intermediate | | | | |
| | colistin-resistant strain | | | | |
| 28 | Salmonella enterica Pennsylvania, Serovar | 50.0 | 1.6 | 1.6 | 1.6 |
| | Typhimurium, Isolate 1 (NR4333) | | | | |
| 29 | Salmonella enterica Pennsylvania, Serovar | 12.5 | >400 | >100 | >100 |
| | Typhimurium, Isolate 1 (NR4333) | | | | |
| | colistin-resistant strain | | | | |
| 30 | Proteus mirabilis urine strain WGLW4 | 3.1 | 200 | >200 | >200 |
| | (Wild type) | | | | |
| 31 | Proteus mirabilis urine strain WGLW4 | 0.8 | >400 | >200 | >200 |
| | colistin-resistant strain | | | | |
| 32 | Serratia sp. strain Ag2 (Wild-type) | 0.8 | 200 | 0.039 | >200 |
| 33 | Serratia sp. strain Ag2 colistin-resistant strain | 0.05 | >400 | 12.5 | >200 |

^{a}All studies were in triplicate. The MIC values were determined via OD and colorimetric assays using risazurin or malachite green.
^{b}Bacteria were purchased from ATCC or acquired from BEI Resources.
^{c}Colistin-resistant strains were prepared in the Kurosu laboratory acording to the procedure described hereinafter.
^{d}Light sources used were a 17 W, 63.8 cm² fluorescent light or a 9.5 W, 28.3 cm² LED light. The MIC was determined after 24 h of treatment (23.0 KJ/cm² for fluorecent light or 29.0 KJ/cm² for LED light). The MIC values obatined under a fluorecent and LED light are the same.

The wild-type E. coli serotype 0157 strain displayed a very low susceptibility against HP-RBf (entry 3), but HP-RBf effectively killed its colistin-resistant mutant (MIC: 3.13 □g/mL) (entry 3 vs. 4). Drug-susceptible and multi-drug-resistant mutants of Acinetobacter baumannii (ATCC19606, entry 5) and A. baumannii (ATCC BAA1800, entry 7) showed resistance to HP-RBf. On the other hand, their colistin-resistant mutants were susceptible to HP-RBf; the MIC value of HP-RBf was 0.05 μg/mL against these two strains (entries 6 and 8). Two wild-type Klebsiella pneumoniae strains (ATCC 19606 and NR48569) were resistant to HP-RBf (MICs>100 μg/mL) (entries 9 and 11), whereas their colistin-resistant mutants increased susceptibility to HP-RBf; the MIC values were 6.3 and 12.5 μg/mL, respectively (entries 10 and 12).

Moreover, we have generated colistin-intermediate (MIC 50 μg/mL) and -high (MIC>400 μg/mL) resistant Pseudomonas aeruginosa mutant with Pseudomonas aeruginosa (ATCC 27853) (entries 14 and 15). The HP-RBf susceptibility of the P. aeruginosa strains was dependent on the degree of its colistin resistance; the MIC values against the higher- and intermediate-colistin resistant strains were 0.05 and 6.3 μg/mL, respectively. Similar to P. aeruginosa (ATCC 27853) studied in Table 1, P. aeruginosa MRSN 1356 (NR51521) and P. aeruginosa MRSN 1380 (NR51522) strains showed low level of intrinsic susceptibility to HP-RBf under illumination conditions (entries 16 and 18) [37]. Their colistin-resistant strain increased the HP-RBf susceptibility (entries 17 and 19).

Some Gram-negative bacteria, such as Burkholderia spp., have been reported to be intrinsically resistant to colistin, with the MIC values often being >200 mg/mL (vide supra). It was realized that a wild type Burkholderia multivorans CGD1 was colistin resistant with a MIC level of 200 mg/mL (Table 1, entry 20 in Table 2).

A colistin-resistant mutant strain of B. multivorans CGD1 was highly susceptible to HP-RBf (entry 21). Similarly, two other Burkholderia spp. (B. thailandensis E264 and B. cepacia genomovar III, LMG16656) were examined; these wild types also showed high colistin-resistance (entries 22 and 24). Their acquired colistin-resistant mutants have 8- and 16-fold increased susceptibility of HP-RBf (entries 23 and 25). Colistin effectively killed most Salmonella spp. with the MIC values of 0.8-1.6 mg/mL (entries 26 and 28).

HP-RBf could inhibit the growth of a wild-type S. enterica subsp. enterica Typhimurium with the MIC value of 12.5 mg/mL (entry 26). A 15-fold higher colistin-resistant mutant of S. enterica subsp. enterica Typhimurium increased its HP-RBf susceptibility (entry 27). An acquired colistin mutant of another Salmonella sp. (S. enterica Pennsylvania, Serovar Typhimurium) also increased its HP-RBf susceptibility (entry 29). Proteus and Serratia spp. are the other bacteria that show resistance to colistin (entries 30 and 32). HP-RBf effectively killed these wild-type strains at low concentrations (entries 30 and 32). As observed in all other entries in Table 2, their acquired colistin mutants increased HP-RBf susceptibility (entries 31 and 32).

### HP-RBf Possesses a Rapid-Killing Feature for Colistin-Resistant Gram-Negative Bacteria

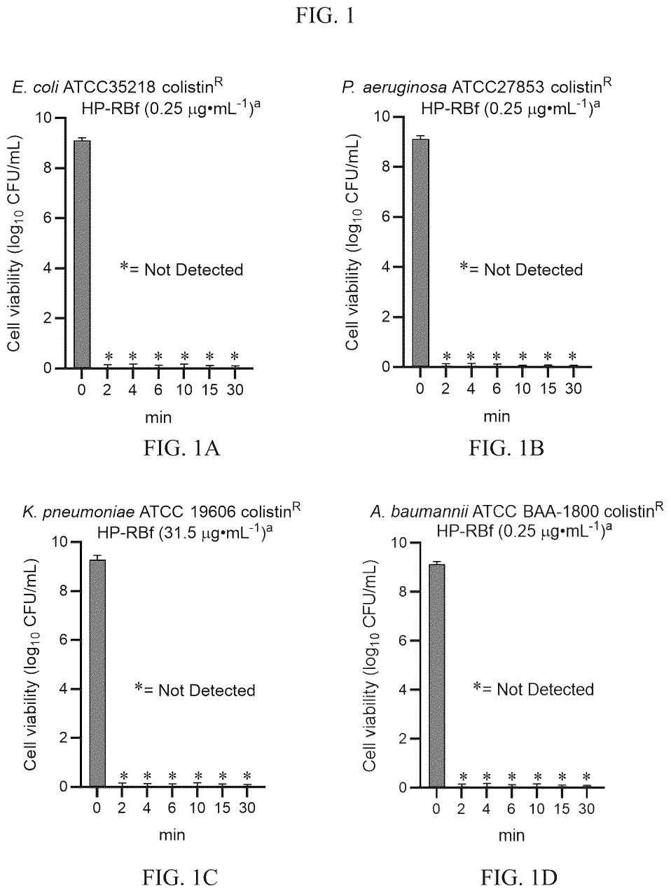

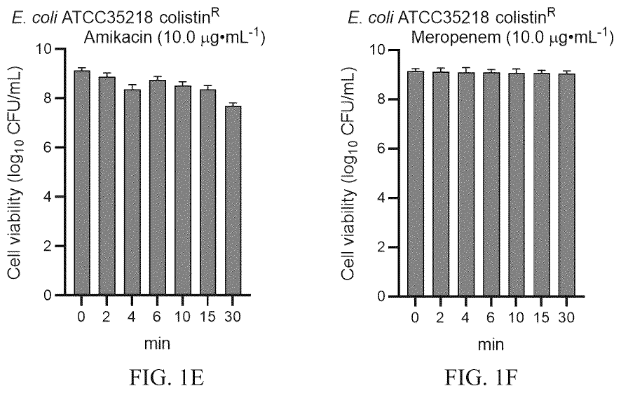

We have reported photodynamic growth inhibitions of HP-RBf against Gram-positive bacteria; the addition of HP-RBf in the bacterial cultures (1.3 to about 4.6×10⁸ CFU/ml) of Gram-positives including drug-resistant strains could cause a greater than a 6-log reduction of bacterial cells in less than 2 minutes (min) [35]. Similarly, the time-course studies on the selected colistin-resistant (colistin^{R}) mutant strains (E. coli ATCC35218 colistin^{R}, P. aeruginosa ATCC27853 colistin^{R}, K. pneumoniae ATCC 19606 colistin^{R}, and A. baumannii ATCC BAA-1800 colistin^{R}) were conducted under a fluorescent light for 24 min (40 J/cm²) at concentrations of 5-fold the MIC (HP-RBf). Graphical illustrations of the results of those are shown in FIG. 1.

Reference molecules used were amikacin (10 mg/mL) and meropenem (10 mg/mL). HP-RBf reduced 1.3 to about 1.9×10⁸ (colony forming units: CFUs) of all colistin-resistant strains by a log reduction of 6 in 2 min; No CFU were counted for the culture media (1×10³ dilution) treated with HP-RBf (0.025 or 31.5 mg/mL) concentrations (FIGS. 1A-1D). In the same studies, amikacin and meropenem reduced about 3.5% (30 min) and 0% (30 min) of E. coli ATCC35218 colistin^{R} strain, respectively (FIGS. 1E and 1D). These results demonstrated that HP-RBf has a fast-killing nature against colistin-resistant Gram-negative bacteria under illumination conditions [35].

### DISCUSSION

We have evaluated the anti-bacterial activity of a pharmaceutical-grade RB-formulated product (HP-RBf) under illumination conditions. We recently reported a comprehensive evaluation of HP-RBf against Gram-positive bacteria and Mycobacterium spp.; over 45 bacterial strains were examined, and HP-RBf displayed a rapid bactericidal activity to Gram-positive bacteria (MIC 0.3-3.1 mg/mL, less than 2 min) [35]. HP-RBf has a strong affinity with Gram-positive peptidoglycan layer and induces photodynamic activation, yielding reactive oxygen species. HP-RBf displays moderate bactericidal activity against Mycobacterial spp. with MIC values of 12.5-25.0 mg/mL (12 hours) under the illumination conditions.

We believe that the mycolic acid-containing thick cell walls reduce the cellular uptake of HP-RBf, in view of the higher MIC values against Mycobacterial spp. than those against Gram-positive bacteria. In previous studies, we concluded that common nosocomial Gram-negative bacteria are not susceptible to HP-RBf; their MIC values are 25 mg/mL or over >50 mg/mL.

We have extended anti-bacterial screening against Gram-negative bacteria and realized that HP-RBf shows susceptibility against some Proteobacteria (Pseudomonadota) such as Burkholderia, some Salmonella, Proteus, and Serratia spp. [21]. The MIC values of HP-RBf are 0.8-12.5 mg/mL against their wild types. One of the characteristics of their drug susceptibility is that they are intrinsically highly resistant to the action of colistin.

HP-RBf killed colostin-resistant mutants of Gram-negative bacteria (E. coli, A. baumannii, K. pneumoniae, and P. aeruginosa) at low concentrations. Enhanced colistin-resistant strains of Burkholderia, Salmonella, Proteus, and Serratia spp. also increased HP-RBf susceptibility. These data unambiguously imply that the acquisition of colistin resistance in Gram-negative bacteria alters the structure of the outer membrane, increasing the affinity of HP-RBf.

It is an established fact that identified colistin-resistance Gram-negative bacteria involve changes of the structure of lipopolysaccharide (LPS) where colistin interacts with the negatively charged lipid A of LPS [39-41]. Thus, increased positively charged LPS components (e.g., cationic forms of 4-amino-L-arabinose, phosphoethanolamine, and galactosamine) in colistin-resistant strains could enhance the affinity of a negatively charged HP-RBf.

We are in the process of further identifying the mode of action for HP-RBF against the colistin-resistant Gram-negative bacteria. Rapid anti-bacterial photodynamic activity of HP-RBf has several advantages, including: 1) lowering the frequency of generation of drug-resistant strains and 2) increasing the safety profile of disinfection and sterilization applications.

In summary, our studies reported here suggest that HP-RBf is a drug candidate for treating intrinsic and acquired colistin-resistant Gram-negative bacterial infections. Colistin-resistant Gram-negative bacteria involve the membrane structure modifications that lead to the resistance of the other important anti-bacterial agents used for Gram-negative bacterial infections [40, 41]. Interestingly, these structural changes make HP-RBf more susceptible to colistin-resistant Gram-negative bacteria.

We reported previously that HP-RBf does not change the integrity of human skin tissue at 200 mM (203.4 mg/mL) concentration in the fluorescent light for 1 hour (h). Therefore, the selectivity index and therapeutic index of HP-RBf for topical application are very high. Our toxicology studies showed that HP-RBf does not have systemic toxicological effects, mutagenic potential, and female reproductive and development effects at therapeutic concentrations [42,43].

To put it all together, HP-RBf is an attractive drug candidate as a rapid bactericidal agent that can be applied to skin, oral, and surgical wound infections. HP-RBf has the potential to serve as a broad-spectrum anti-bacterial agent for Gram-positive bacteria (previous studies) and Gram-negative bacteria (this study) in combination with an anti-Gram-negative drug.

### Materials and Methods

### General/Chemicals and Reagents

All chemicals and antibiotics were purchased from commercial sources, including Sigma-Aldrich, and used without further purification unless otherwise noted. High-purity rose bengal was synthesized by the team at Provectus Biopharmaceuticals, Inc. (USA). All bacterial culture media used for growth inhibitory activity tests were purchased from Fisher Scientific. A fluorescent light [17 W, 63.8 cm², Sunblaster Holdings, ULC (Langly B. C., CA)] and LED (9.5 W, 28.3 cm², Philips) were purchased from Amazon.com. Resazurin (Alamar blue) was purchased from Sigma-Aldrich.

### Bacterial Strains

All bacteria studied in this project were purchased or acquired from American Type Culture Collection (ATCC) or BEI Resources (NIAID), including E. coli (ATCC 35218), E. coli serotype 0157 (TW07793), A. baumannii (ATCC19606), A. baumannii (ATCC BAA1800), K. pneumoniae (ATCC 19606), K. pneumoniae (CRE) CHS67 (NR48569), K. pneumoniae VA360 (NR48977), P. aeruginosa (ATCC27853), P. aeruginosa MRSN 1356 (NR51521), P. aeruginosa MRSN 1380 (NR51522), S. Typhimurium (ATCC BAA 2721), S. enterica serovar Typhimurium (NR4333), Burkholderia cepacian (UCB717), and S. pneumoniae (ATCC6301). Colistin-resistant strains were generated by the method described below.

### Formulation of Pharmaceutical-Grade Rose Bengal in Saline (HP-RBf).

Rose bengal disodium salt formulation (10%) in saline (HP-RBf) was provided by Provectus Biopharmaceuticals, Inc. (Knoxville, TN, USA). HP-RBf (>99.5%) was synthesized according to Provectus' proprietary process to synthesize and utilize the RB molecule as a viable drug substance for commercial use. The detailed procedure was previously described in [35], and in the previously noted U.S. patents to U.S. Pat. Nos. 8,530,675, 9,273,022, and 9,422,260 to Singer et al.

### Log Phase Bacterial Culture

All liquid bacterial culturing was performed in a conical flask with an air filter. A single colony of a bacterial strain was grown under recommended conditions suggested by ATCC. The culture flasks were incubated for 24 hours (h) in a shaking incubator at 37° C. with a shaking speed of 200 rpm and cultured to mid-log phase (optical density-0.5). The optical density was monitored at 600 nm using a 96 well microplate reader.

### Determination of Minimum Inhibitory Concentration (MIC)

All assays were conducted by following the guidelines set by the Clinical & Laboratory Standards Institute (CLSI; Wayne, PA, USA). Minimum inhibitory concentrations (MICs) were determined by broth dilution microplate alamar blue assay or by OD measurement. All commercial compounds were stored in DMSO or saline (1 mg/100 μL concentration). 10% Rose bengal disodium salts formulation in saline (HP-RBf) was diluted with saline to form 1 mg/100 □L stock solution. Stock solution aliquots were stored at 4° C. for the duration of the experiments.

Each compound from the stock solution was placed in the first well of a sterile 96 well plate, and a serial dilution was conducted with the culturing broth (total volume of 10 μL). The bacterial suspension at log phase (190 μL) was placed in each well (total volume of 200 μL), treated with serial dilutions of anti-bacterial agents in aerobic conditions, and incubated at 37° C. for 24 h.

MIC studies for HR-RBf were performed under a fluorescent light (17 W, 63.8 cm², Sunblaster Holdings) and LED (9.5 W, 28.3 cm², Philips). ODs were measured via a UV-Vis spectrophotometer. Resazurin (20 μL) was incubated on a shaking incubator for 2 hours at 37° C. See National Committee for Clinical Laboratory Standards [(NCCLS); method (pink=growth, blue=no visible growth)]. The OD measurements were performed for all studies prior to performing colorimetric assays. The absorbance measurements were done using a Biotek Synergy XT, 96 well plate reader at 570 nm and 600 nm.

### Generation of Drug-Resistant Gram-Negative Bacterial Strains

Colistin-resistant mutants of tested bacterial strains were generated according to the same procedure. A bacterial culture (100 μL of 1×10⁷ CFU/mL) was plated on an agar plate (55 cm²) containing colistin [minimum bactericidal concentration (MBC)]. The colonies grown on the colistin-containing agar plate were collected and suspended in a PBS buffer (about 1×10⁷ CFU/mL), and 100 □L of the bacterial suspension was plated on the agar plate containing antibiotics (1.5×MBC). This process was repeated until the cells acquired >10 times higher MIC level than the wild type; the concentrations of antibiotic were gradually increased (2.0×, 2.5×, 3.0×, 3.5×, 4.0×, 5.0×, 7.0×, 8.0×, 9.0×, 10.0×, 20×, and 50× MBC). The isolated resistant cells were confirmed by the MIC assay with the generated resistant strain.

### Time-Kill Kinetic Assays

Time-kill kinetics assay for antimicrobial agents was performed based on the CLSI guidelines with a minor modification. The multiple time points in time-kill kinetics assays for HP-RBf and the reference molecules were performed. The bacterial culture grown in the broth was diluted to a concentration between 1×10⁸ and 5.0×10⁹ CFU/mL with a stock dilution of HP-RBf, which was prepared at 5-fold the MIC values.

The test compounds were inoculated with equal volumes of the specified bacteria placed in a 96-well plate. The microtiter plates were incubated at 37° C. and duration (1-30 min) under the fluorescent light (conditions are summarized in the above Table legends). An aliquot of the culture medium was taken from each well, and a serial dilution was performed. The diluted culture was incubated at 37° C., and CFU/mL were counted. Bactericidal activity was defined as greater than a 3 log-fold decrease in colony-forming units.

### CITATIONS

1. Boucher, H. W., Talbot, G. H., Benjamin, D. K., Bradley, J., Guidos, R. J., Jones, R. N., Murray, B. E., Bonomo, R. A., Gilbert, D. New drugs for Gram-negative bacilli. Clin. Infect. Dis. 56, 1685-1693 (2013).
2. Bassetti, M., Peghin, M., Vena, A., Giacobbe, D. R. Treatment of infections due to MDR Gram-negative bacteria. Front. Med. (Lausanne). 16, 74 (2019).
3. Bassetti, M., Vena, A., Giacobbe, D. R., Castaldo, N. Management of infections caused by multidrug-resistant Gram-negative pathogens: Recent advances and future directions. Arch. Med. Res. 52, 817-827 (2021).
4. Oliveira, J., Reygaert, W. C. Gram negative bacteria. 2022 Oct. 8. In: StatPearls [Internet]. Treasure Island (FL): StatPearls Publishing; 2022 January. PMID: 30855801.
5. Loho, T., Dharmayanti, A. Colistin: an antibiotic and its role in multiresistant Gram-negative infections. Acta Med. Indones. 47, 157-68 (2015).
6. Gharaibeh, M. H., Shatnawi, S. Q. An overview of colistin resistance, mobilized colistin resistance genes dissemination, global responses, and the alternatives to colistin: A review. Vet. World. 12, 1735-1746 (2019).
7. Pike, M., Saltiel, E. Colistin- and polymyxin-induced nephrotoxicity: focus on literature utilizing the RIFLE classification scheme of acute kidney injury. J. Pharm. Pract. 27, 554-561 (2014).
8. Ordooei, Javan. A., Shokouhi, S., Sahraei, Z. A review on colistin nephrotoxicity. Eur. J. Clin. Pharmacol. 71, 801-810 (2015).
9. Jafari, F., Elyasi, S. Prevention of colistin induced nephrotoxicity: a review of preclinical and clinical data. Expert Rev. Clin. Pharmacol. 14, 1113-1131 (2021).
10. Eljaaly, K., Bidell, M. R., Gandhi, R. G., Alshehri, S., Enani, M. A., Al-Jedai, A., Lee, T. C. Colistin nephrotoxicity: Meta-analysis of randomized controlled trials. Open Forum Infect. Dis. 8 (2021).
11. Biswas, S., Brunel, J. M., Dubus, J. C., Reynaud-Gaubert, M., Rolain, J. M. Colistin: an update on the antibiotic of the 21st century. Expert Rev. Anti. Infect. Ther. 10, 917-934 (2021).
12. Qadi M, Alhato S, Khayyat R, Elmanama AA. Colistin Resistance among Enterobacteriaceae Isolated from Clinical Samples in Gaza Strip. Can J Infect Dis Med Microbiol. 2021 Apr. 20; 2021:6634684.
13. Aghapour, Z., Gholizadeh, P., Ganbarov, K., Bialvaei, A. Z., Mahmood, S. S., Tanomand, A., Yousefi, M., Asgharzadeh, M., Yousefi, B., Kafil, H. S. Molecular mechanisms related to colistin resistance in Enterobacteriaceae. Infect. Drug. Resist. 24, 965-975 (2019).
14. Zafer, M. M., El-Mahallawy, H. A., Abdulhak, A., Amin, M. A., Al-Agamy, M. H., Radwan, H. H. Emergence of colistin resistance in multidrug-resistant Klebsiella pneumoniae and Escherichia coli strains isolated from cancer patients. Ann. Clin. Microbiol. Antimicrob. 18, 40 (2019).
15. Azimi, L., Lari, A. R. Colistin-resistant Pseudomonas aeruginosa clinical strains with defective biofilm formation. GMS Hyg. Infect. Control. 10 (2019).
16. Bialvaei, A. Z., Kafil H. S. Colistin, mechanisms and prevalence of resistance. Curr. Med. Res. Opin. 31, 707-721 (2015).
17. Qureshi, Z. A., Hittle, L. E., O'Hara, J. A., Rivera, J. I., Syed, A., Shields, R. K., Pasculle, A. W., Ernst, R. K., Doi, Y. Colistin-resistant Acinetobacter baumannii: beyond carbapenem resistance. Clin. Infect. Dis. 60, 1295-1303 (2015).
18. Humphrey, M., Larrouy-Maumus, G. J., Furniss, R. C. D., Mavridou, D. A. I., Sabnis, A., Edwards, A. M. Colistin resistance in Escherichia coli confers protection of the cytoplasmic but not outer membrane from the polymyxin antibiotic. Microbiology (Reading) 167, 001104 (2021).
19. Lima, T., Domingues, S., Da Silva, G. J. Plasmid-mediated colistin resistance in Salmonella enterica: A review. Microorganisms 19, 55 (2019).
20. Janssen, A. B., Doorduijn, D. J., Mills, G., Rogers, M. R. C., Bonten, M. J. M., Rooijakkers, S. H. M., Willems, R. J. L., Bengoechea, J. A., van Schaik, W. Evolution of colistin resistance in the Klebsiella pneumoniae complex follows multiple evolutionary trajectories with variable effects on fitness and virulence characteristics. Antimicrob. Agents Chemother. 16, e01958-20 (2020).
21. Gogry, F. A., Siddiqui, M. T., Sultan, I., Haq, Q. M. R. Current update on intrinsic and acquired colistin resistance mechanisms in bacteria. Front. Med. (Lausanne) 12, 677720 (2021).
22. Napier, B. A., Burd, E. M., Satola, S. W., Cagle, S. M., Ray, S. M., McGann, P., Pohl, J., Lesho, E. P., Weiss, D. S. Clinical use of colistin induces cross-resistance to host antimicrobials in Acinetobacter baumannii. mBio. 21, e00021-13 (2013).
23. Dobias, J., Poirel, L., Nordmann, P. Cross-resistance to human cationic antimicrobial peptides and to polymyxins mediated by the plasmid-encoded MCR-1? Clin. Microbiol. Infect. 23, 676.e1-676.e5 (2017).
24. Hirsch, H. A., Mccarthy, C. G., Finland, M. Polymyxin B and colistin; activity, resistance, and cross-resistance in vitro. Proc. Soc. Exp. Biol. Med. 103, 338-342 (1960).
25. Livermore, D. M., Mushtaq, S., Warner, M. Selectivity of ertapenem for Pseudomonas aeruginosa mutants cross-resistant to other carbapenems. J. Antimicrob. Chemother. 55, 306-311 (2005).
26. Petrosillo, N., Taglietti, F., Granata, G. Treatment options for colistin resistant Klebsiella pneumoniae: Present and future. J. Clin. Med. 28, 934 (2019).
27. Jana, B., Cain, A. K., Doerrler, W. T., Boinett, C. J., Fookes, M. C., Parkhill, J., Guardabassi, L. The secondary resistome of multidrug-resistant Klebsiella pneumoniae. Sci. Rep. 15, 42483 (2017).
28. Boucher, H. W., Talbot, G. H., Benjamin, D. K., Bradley, J., Guidos, R. J., Jones, R. N., Murray, B. E., Bonomo, R. A., Gilbert, D. New drugs for Gram-negative bacilli. Clin. Infect. Dis. 56, 1685-1693 (2013).
29. Meyer, A. L. Prospects and challenges of developing new agents for tough Gram-negatives. Curr. Opini. Pharmacol. 5, 490-494 (2005).
30. El-Gawad El-Sayed Ahmed, M. A., Zhong, L-L., Shen, C., Yang, Y., Doi, Y., Tian, G-B. Colistin and its role in the Era of antibiotic resistance: an extended review (2000-2019). Emerg. Microbes. Infect. 9, 868-885 (2020).
31. Aris, P., Robatjazi, S., Nikkhahi, F., Marashi, S. M. A. Molecular mechanisms and prevalence of colistin resistance of Klebsiella pneumoniae in the Middle East region: A review over the last 5 years. J. Glob. Antimicrob. Resist. 22, 625-630 (2020).
32. Zhang, Q., Chen, S., Liu, X., Lin, W., Zhu, K. Equisetin restores colistin sensitivity against multi-drug resistant Gram-negative bacteria. Antibiotics (Basel) 18, 1263 (2021).
33. Elio, C., PhD; Erica, R., Marcello, M. Infections due to antibiotic-resistant Gram-negative bacteria in pediatrics: Possible management strategies. Pediatric Infect. Dis. J. 41, e283-e285 (2022).
34. Lelovic, N., Mitachi, K., Yang, J., Lemieux, M. R., Ji, Y., Kurosu, M. Application of Mycobacterium smegmatis as a surrogate to evaluate drug leads against Mycobacterium tuberculosis. J. Antibiotics 73, 780-789 (2020).
35. Kurosu, M., Mitachi, K., Yang, J., Pershing, E. V., Horowitz, B. D., Wachter, E. A., Lacey, J. W. 3^{rd}., Ji Y., Rodrigues, D. J. Anti-bacterial activity of pharmaceutical-grade rose bengal: An application of a synthetic dye in anti-bacterial therapies. Molecules 27, 322 (2022).
36. Patel, S. P., Carter, B. W. Percutaneous hepatic injection of rose bengal disodium (PV-10) in metastatic uveal melanoma. J. Clin. Oncol. 38, 3143 (2020).
37. Nakonieczna, J., Wolnikowska, K. Rose bengal-mediated photoinactivation of multidrug resistant Pseudomonas aeruginosa is enhanced in the presence of antimicrobial peptides. Front. Microbiol. 9, 1949 (2018).
38. Zhou, K., Cattoir, V., Xiao, Y. Intrinsic colistin resistance. Lancet Infect. Dis. 16, 1227-1228 (2016).
39. Li, X., Gu, Y., Dong, H., Wang, W., Dong, C. Trapped lipopolysaccharide and LptD intermediates reveal lipopolysaccharide translocation steps across the Escherichia coli outer membrane. Sci. Rep. 7, 11883 (2015).
40. Sabnis, A., Hagart, K. L., Klöckner, A., Becce, M., Evans, L. E., Furniss, R. C. D., Mavridou, D. A., Murphy, R., Stevens, M. M., Davies, J. C., Larrouy-Maumus, G. J., Clarke, T. B., Edwards, A. M. Colistin kills bacteria by targeting lipopolysaccharide in the cytoplasmic membrane. Elife 6, e65836 (2021).
41. Freinkman, E., Chng, S-S., Kahne, D. The complex that inserts lipopolysaccharide into the bacterial outer membrane forms a two-protein plug-and-barrel. PNAS 108, 2486-2491 (2010).
42. Wachter, E., Dees, C., Harkins, J., Scott, T., Petersen, M., Rush, R. E., Cada, A. Topical rose bengal: pre-clinical evaluation of pharmacokinetics and safety. Lasers Surg. Med. 32, 101-10 (2003).
43. Lee, Y. C., Park, C. K., Kim, M. S., Kim, J. H. In vitro study for staining and toxicity of rose bengal on cultured bovine corneal endothelial cells. Cornea 15, 376-385 (1996).

1. A method of treating a Gram-negative bacteria that also exhibit resistance to the anti-bacterial compound colistin other than Burkholderia, Proteus, and Serratia that comprises the steps of:
a) contacting said Gram-negative bacteria with an aqueous pharmaceutical composition containing a rose bengal (RB) compound of Formula I, below, dissolved or dispersed therein at a concentration of about 0.01 to about 15 mg/mL; and
b) irradiating those contacted bacteria with light of the wavelength about 500 nm to about 600 nm for a time period of about 1 to about 10 minutes to provide a light dose of about 16 to about 160 J/cm², irradiated bacteria, 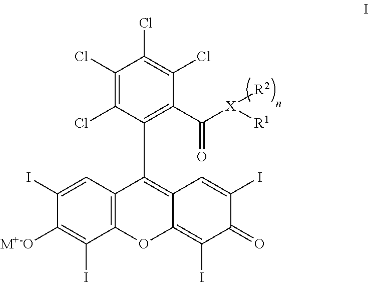
wherein X is oxygen or nitrogen, “n” is zero or 1 such that when X is oxygen, n is zero and R² is absent, and when X is nitrogen, n is 1 and R² is present;
when X is oxygen, R¹ is selected from the group consisting of hydrogen (H), M⁺ that is a pharmaceutically acceptable cation, C₁-C₄ alkyl, and an aromatic ring as defined herein after;
when X is nitrogen, R¹ and R² are the same or different and are selected from the group consisting of hydrogen, C₁-C₄ alkyl, or together with amido nitrogen atom form a 5- or 6-membered ring, and an aromatic ring as defined herein after;
wherein said aromatic ring is a single ring containing 5- or 6-members, or a 5,6- or 6, 6-fused aromatic ring system, said aromatic ring or ring system containing 0, 1 or 2 hetero ring atoms that are independently nitrogen, oxygen or sulfur.

2. The method according to claim 1, wherein said RB compound is rose bengal disodium.

3. The method according to claim 1, wherein said Gram-negative colistin-resistant bacteria are one or more of E. coli, A. baumannii, K. pneumoniae, P. aeruginosa, and S. enterica.

4. The method according to claim 1, wherein said Gram-negative colistin-resistant bacteria are present within or on mammalian cells when contacted.

5. The method according to claim 1, wherein said Gram-negative colistin-resistant bacteria are present on or in a surgical or other wound of mammalian subject.

6. The method according to claim 1, wherein said Gram-negative colistin-resistant bacteria are present on one or more of an examination table, a floor, a wall, and an equipment of a surgical arena.

7. The method according to claim 1, wherein said Gram-negative colistin-resistant bacteria are irradiated for a time period of about 2 to about 5 minutes to provide a light dose of about 32 to about 80 J/cm².

8. The method according to claim 1, wherein said aromatic ring substituent is selected from one or more of the group consisting of one or more of 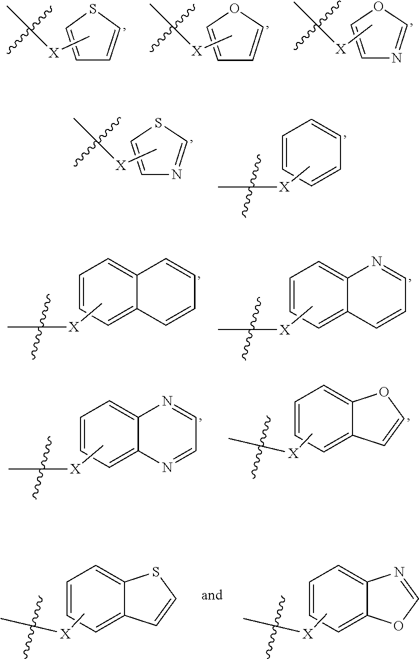
where 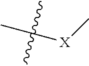
is 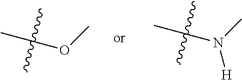
providing an ester or a monosubstituted amine, respectively.

9. A pharmaceutical composition for treating a Gram-negative bacteria that also exhibits resistance to an anti-bacterial compound colistin other than Burkholderia, Proteus, and Serratia, containing a rose bengal (RB) compound of Formula I, below, 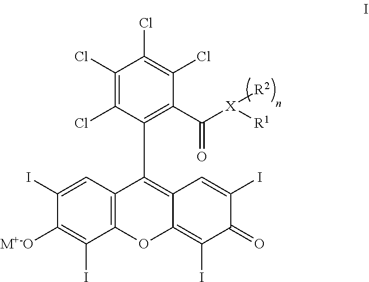
wherein X is oxygen or nitrogen, “n” is zero or 1 such that when X is oxygen, n is zero and R² is absent, and when X is nitrogen, n is 1 and R² is present;
when X is oxygen, R¹ is selected from the group consisting of hydrogen (H), M⁺ that is a pharmaceutically acceptable cation, C₁-C₄ alkyl, and an aromatic ring as defined herein after;
when X is nitrogen, R¹ and R² are the same or different and are selected from the group consisting of hydrogen, C₁-C₄ alkyl, or together with amido nitrogen atom form a 5- or 6-membered ring, and an aromatic ring as defined herein after;
wherein said aromatic ring is a single ring containing 5- or 6-members, or a 5,6- or 6, 6-fused aromatic ring system, said aromatic ring or ring system containing 0, 1 or 2 hetero ring atoms that are independently nitrogen, oxygen or sulfur.

10. The composition of claim 9, wherein said RB compound is rose bengal disodium.

11. The composition of claim 9, wherein said RB compound has a pH value of 6.5 to 7.4.

12. The composition of claim 9, wherein said RB compound has a pKa value of 2.52 or 1.81.

13. The composition of claim 9, wherein said composition further comprises a water-soluble electrolyte comprising at least one cation selected from the group consisting of sodium, potassium, calcium, and magnesium and at least one anion selected from the group consisting of chloride, phosphate, and nitrate.

14. The composition of claim 13, wherein the water-soluble electrolyte is sodium chloride.

15. The composition of claim 13, wherein the water-soluble electrolyte is present in the composition at a concentration of 0.1 weight percent by volume to 2 weight percent by volume.

16. The composition of claim 13, wherein the water-soluble electrolyte is present in the composition at a concentration sufficient to provide an osmolality of 300 milliosmoles per kilogram of water to 500 milliosmoles per kilogram of water.

17. The composition of claim 9, wherein said composition further comprises a diluent including a C₁-C₆ mono- or polyhydric alcohol present in the diluent at a concentration of less than 5 percent by volume.

18. The aqueous composition of claim 9, wherein said aqueous composition is provided in the form of a liquid, a gel, or a cream.

19. A bactericidal agent for treating a Gram-negative bacteria that also exhibits resistance to an anti-bacterial compound colistin other than Burkholderia, Proteus, and Serratia, containing a rose bengal (RB) compound of Formula I, below, 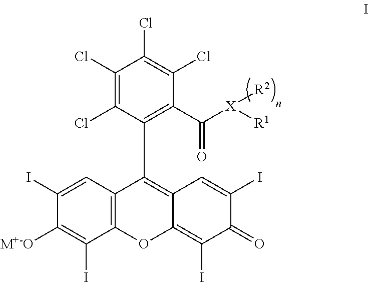
wherein X is oxygen or nitrogen, “n” is zero or 1 such that when X is oxygen, n is zero and R² is absent, and when X is nitrogen, n is 1 and R² is present;
when X is oxygen, R¹ is selected from the group consisting of hydrogen (H), M⁺ that is a pharmaceutically acceptable cation, C₁-C₄ alkyl, and an aromatic ring as defined herein after;
when X is nitrogen, R¹ and R² are the same or different and are selected from the group consisting of hydrogen, C₁-C₄ alkyl, or together with amido nitrogen atom form a 5- or 6-membered ring, and an aromatic ring as defined herein after;
wherein said aromatic ring is a single ring containing 5- or 6-members, or a 5,6- or 6, 6-fused aromatic ring system, said aromatic ring or ring system containing 0, 1 or 2 hetero ring atoms that are independently nitrogen, oxygen or sulfur,
wherein said bactericidal agent is designed to be used in combination with a fluorescent light source with a wavelength about 500 nm to about 600 nm,
wherein said light source is applied to the Gram-negative bacteria for a time period of about 1 to about 10 minutes to provide a light dose of about 16 to about 160 J/cm².

20. The bactericidal agent of claim 19, wherein said bactericidal agent is applied to at least one of a surface of a surgical area, a surgical instrument, a surgeon's hands, and intracorporeal area of a patient's body, and an extracorporeal area of a surgical patient's body.
